(19) United States
(12) Patent Application Publication
(10) Pub. No.: US 2026/0098301 A1
(43) Pub. Date: Apr. 9, 2026

(54) METHODS AND SYSTEMS FOR PROCESSING GENETIC VARIATIONS AND PHENOTYPES

(71) Applicant: GeneDx, LLC, Gaithersburg, MD (US)

(72) Inventors: Amber Begtrup, Cincinnati, OH (US); Deanna Alexis Carere, Delaware (CA); Amanda Lindy, Rockville, MD (US); Maria Guillen Sacoto, Natick, MA (US)

(21) Appl. No.: 19/183,496

(22) Filed: Apr. 18, 2025

Related U.S. Application Data
(60) Provisional application No. 63/704,757, filed on Oct. 8, 2024.
(60) Provisional application No. 63/717,540, filed on Nov. 7, 2024.
(60) Provisional application No. 63/722,378, filed on Nov. 19, 2024.
(60) Provisional application No. 63/736,437, filed on Dec. 19, 2024.
(60) Provisional application No. 63/769,928, filed on Mar. 11, 2025.

(51) Int. Cl. C12Q 1/6883; C12Q 1/6874

(52) U.S. Cl. CPC C12Q 1/6883; C12Q 1/6874; C12Q 2600/156

(57) ABSTRACT

The present disclosure provides a systems and methods for processing genetic variations and phenotypic data. The systems and methods may be used to generate one or more databases comprising genetic variations and associations with phenotypes. The system and methods may be used to determine a pathogenicity of a genetic variation. The systems and methods may comprise human interpretation using pre-determined criteria.

### CROSS-REFERENCE

This application claims the benefit of U.S. Provisional Application No. 63/704,757, filed Oct. 8, 2024, U.S. Provisional Application No. 63/717,540, filed Nov. 7, 2024, U.S. Provisional Application No. 63/722,378, filed Nov. 19, 2024, U.S. Provisional Application No. 63/736,437, filed Dec. 19, 2024, and U.S. Provisional Application No. 63/769,928, filed Mar. 11, 2025, each of which are incorporated herein by reference in their entirety.

### BACKGROUND

Diagnostic genetic testing may involve examining a subject's DNA to detect genetic variants that may cause illness or disease. Diagnostic genetic testing may play a fundamental role in determining an individual's risk of developing certain diseases and uncovering genetic causes of illnesses that a subject may be suffering from.

### SUMMARY

The present disclosure recognizes a need for improved methods for analyzing relationships between a subject's clinical symptoms and their genetic profile.

Aspects disclosed herein provide methods for (a) assaying a bodily sample from a subject to generate sequencing information; (b) computer processing the sequencing information using a gene-phenotype knowledge base to identify a prospective genetic variation and a phenotype associated with the prospective genetic variation; (c) determining a pathogenicity of the prospective genetic variation, wherein determining the pathogenicity comprises computer processing the prospective genetic variation and the phenotype identified in (b); and (d) generating a human interpretation of a clinical significance of the pathogenicity determined in (c), wherein the human interpretation comprises applying a set of pre-determined criteria to the determining in (c).

In some embodiments, the bodily sample is a buccal sample, a blood sample, a saliva sample, a urine sample, a cell sample, or a tissue sample. In some embodiments, (a) comprises extracting DNA from the bodily sample.

In some embodiments, the method further comprises preparing a sequencing library from the DNA. In some embodiments, (a) comprises subjecting the DNA to a sequencing reaction. In some embodiments, the sequencing reaction comprises a whole genome sequencing reaction, or an exome sequencing reaction, or both.

In some embodiments, the method further comprises subjecting the DNA, or derivatives thereof to one or more pull down probes. In some embodiments, the subject is less than 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, 3 years old, 2 years old, or 1 year old. In some embodiments, the method further comprises obtaining data relating to the phenotype of the subject is based at least in part on medical records or clinical notes. In some embodiments, the prospective genetic variation comprises one or more of: a substitution, a insertion, a deletion, a single nucleotide variation, a copy number variation, a mobile element insertion (MEI), or a Uniparental disomy (UPD), or any combination thereof.

In some embodiments, the method further comprises prior to (c), performing an orthogonal assay to confirm a presence of the prospective genetic variation. In some embodiments, the orthogonal assay comprises a chromosomal microarray analysis (CMA), exon-level ‘exon-array’ microarray, qPCR, multiplex ligation-dependent probe amplification (MLPA), or a Sanger sequencing assay. In some embodiments, the prospective genetic variation is classified as likely benign, benign, likely pathogenic, pathogenic, or uncertain significance. In some embodiments, a genetic variation classified as likely benign indicates at least about a 90% certainty of benignity. In some embodiments, a variant classified as benign indicates at least about a 99% certainty of benignity. In some embodiments, a variant classified as likely pathogenic indicates at least about a 90% certainty of pathogenicity. In some embodiments, a variant classified as pathogenic indicates at least about a 99% certainty of pathogenicity. In some embodiments, the processing comprises using a trained machine learning algorithm. In some embodiments, the processing outputs one or more scores indicative of strength of association of the prospective genetic variation with the phenotype associated with the prospective genetic variation. In some embodiments, the gene-phenotype knowledge base comprises data derived from two or more related individuals, or published literature, or both. In some embodiments, the set of pre-determined criteria is based at least at least in part on one or more of: a frequency of the prospective genetic variation in affected and unaffected populations of a disease or disorder, a variant type, a disease mechanism, segregation patterns of one or more loci, analysis of functional studies, analysis of case studies, or analysis of cohort studies, or any combination thereof. In some embodiments, the set of pre-determined criteria is based at least upon data from the subject and a data from a mother or father of the subject. In some embodiments, the method is performed in less than 7 days. In some embodiments, the method is performed in less than 10 days. In some embodiments, the method is performed with an accuracy of at least about 90%. In some embodiments, the human interpretation further confirms the phenotype associated with the prospective genetic variation.

In some embodiments, the method further comprises, based at least on the presence of the prospective genetic variation and the phenotype associated with the prospective genetic variation, determining that the subject has an elevated risk of having a disease, disorder, or condition. In some embodiments, determining that the subject has the elevated risk of having the disease, disorder, or condition comprises a sensitivity of at least about 90%. In some embodiments, determining that the subject has the elevated risk of having the disease, disorder, or condition comprises a specificity of at least about 90%.

Aspects disclosed herein provide methods for (a) obtaining training information comprising a set of genes and a set of phenotypes; (b) computer processing the training information to identify a prospective genetic variation and a phenotype associated with the prospective genetic variation; (c) generating a human interpretation of a clinical significance of an association between the prospective genetic variation and the phenotype, wherein the human interpretation comprises applying a set of pre-determined criteria to the identifying in (b); and (d) incorporating the association into a gene-phenotype knowledge base, based at least in part on the human interpretation in (c).

Aspects disclosed herein provide methods for (a) assaying a bodily sample from a subject to generate sequencing information; (b) computer processing the sequencing information using a gene-phenotype knowledge base to identify a prospective genetic variation and a phenotype associated with the prospective genetic variation; (c) determining a pathogenicity of the prospective genetic variation, wherein determining the pathogenicity comprises computer processing the prospective genetic variation and the phenotype identified in (b); and (d) generating a human interpretation of a clinical significance of the pathogenicity determined in (c), wherein the human interpretation comprises applying a set of pre-determined criteria to the determining in (c).

In some embodiments, the bodily sample is a buccal sample, a blood sample, a saliva sample, a urine sample, a cell sample, or a tissue sample. In some embodiments, the bodily sample is a blood sample.

In some embodiments, the method further comprises extracting DNA from the bodily sample. In some embodiments, the method further comprises preparing a sequencing library from the DNA. In some embodiments, the method further comprises preparing a sequencing library comprises shearing the DNA. In some embodiments, the preparing the sequencing library comprises ligating adapters to the DNA, or derivatives thereof. In some embodiments, the preparing the sequencing library comprises amplifying the DNA.

In some embodiments, the method further comprises subjecting the DNA to a sequencing reaction. In some embodiments, the sequencing reaction comprises a whole genome sequencing reaction. In some embodiments, the sequencing reaction is a exome sequencing reaction. In some embodiments, the method further comprises subjecting the DNA, or derivatives thereof to one or more pull down probes.

In some embodiments, the subject is a fetus or a newborn. In some embodiments, the subject is less than 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, 3 years old, 2 years old, or 1 year old. In some embodiments, the subject is an adult.

In some embodiments, the method further comprises obtaining data relating to a medical records or clinical notes. In some embodiments, the phenotype of the subject is based at least upon the medical records or the clinical notes.

In some embodiments, the prospective genetic variation comprises a substitution, insertion, or deletion. In some embodiments, the prospective genetic variation comprises single nucleotide variation, copy number variation. In some embodiments, the prospective genetic variation comprises a repeat expansion, a mobile element insertion (MEI), or a Uniparental disomy (UPD). In some embodiments, the prospective genetic variation is classified as likely benign, benign, likely pathogenic, pathogenic, or uncertain significance. In some embodiments, a genetic variation classified as likely benign indicates at least about a 90% certainty of benignity. In some embodiments, a variant classified as benign indicates at least about a 99% certainty of benignity. In some embodiments, a variant classified as likely pathogenic indicates at least about a 90% certainty of pathogenicity. In some embodiments, a variant classified as pathogenic indicates at least about a 99% certainty of pathogenicity.

In some embodiments, the method further comprises, prior to determining a pathogenicity of the prospective genetic variation, performing an orthogonal assay to confirm a presence of the prospective genetic variation. In some embodiments, the orthogonal assay comprises a chromosomal microarray analysis (CMA), exon-level ‘exon-array’ microarray, qPCR, multiplex ligation-dependent probe amplification (MLPA), or a Sanger sequencing assay.

In some embodiments, the processing comprises using a trained machine learning algorithm. In some embodiments, the processing outputs one or more scores indicative of strength of association of the prospective genetic variation with the phenotype associated with the prospective genetic variation. In some embodiments, the human interpretation further confirms the phenotype associated with the prospective genetic variation. In some embodiments, the method further comprises, based at least on the presence of the prospective genetic variation and the phenotype associated with the prospective genetic variation, determining that the subject has an elevated risk of having a disease, disorder, or condition. In some embodiments, the method further comprises determining that the subject has the elevated risk of having the disease, disorder, or condition comprises a sensitivity of at least about 90%. In some embodiments, the method further comprises determining that the subject has the elevated risk of having the disease, disorder, or condition comprises a specificity of at least about 90%.

In some embodiments, the gene-phenotype knowledge base comprises data derived from published literature. In some embodiments, the gene-phenotype knowledge bases comprises data derived from Online Mendelian Inheritance in Man, Orphanet, GeneReviews, or a combination thereof. In some embodiments, the gene-phenotype knowledge base comprises data derived from two or more related individuals.

In some embodiments, the set of pre-determined criteria is based at least upon a frequency of the prospective genetic variation in affected and unaffected populations of a disease or disorder. In some embodiments, the set of pre-determined criteria is based at least upon a variant type. In some embodiments, the set of pre-determined criteria is based at least upon a disease mechanism. In some embodiments, the set of pre-determined criteria is based at least upon segregation patterns of one or more loci. In some embodiments, the set of pre-determined criteria is derived at least in part from analysis of functional studies, case studies, or cohort studies. In some embodiments, the set of pre-determined criteria is based at least upon data from the subject and a data from a mother or father of the subject.

In some embodiments, the method is performed in less than 7 days. In some embodiments, the method is performed in less than 10 days. In some embodiments, the method is performed with an accuracy of at least about 90%.

Aspects disclosed herein provide methods for (a) obtaining training information comprising a set of genes and a set of phenotypes, (b) computer processing the training information to identify a prospective genetic variation and a phenotype associated with the prospective genetic variation, (c) generating a human interpretation of a clinical significance of an association between the prospective genetic variation and the phenotype, wherein the human interpretation comprises applying a set of pre-determined criteria to the identifying in (b), and (d) incorporating the association into a gene-phenotype knowledge base, based at least in part on the human interpretation in (c).

In some embodiments, the training information comprises information derived from sequencing one or more individuals. In some embodiments, the sequencing comprises a whole genome sequencing. In some embodiments, the sequencing comprises an exome sequencing reaction. In some embodiments, the sequencing comprises a targeted sequencing reaction. In some embodiments, the targeted sequencing reaction comprises using one or more pull down probes.

In some embodiments, the method further comprises, prior to computer processing the training information to identify a prospective genetic variation and a phenotype associated with the prospective genetic variation, obtaining data relating to a medical records or clinical notes corresponding to one or more individuals. In some embodiments, one or more phenotypes of the one or more individuals is identified based at least upon the medical records or the clinical notes.

In some embodiments, one or more individuals comprise a fetus or a newborn. In some embodiments, one or more individuals comprise an individual younger than 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, 3 years old, 2 years old, or 1 year old. In some embodiments, one or more individuals comprise an adult.

In some embodiments, the prospective genetic variation comprises a substitution, insertion, or deletion. In some embodiments, the prospective genetic variation comprises single nucleotide variation, copy number variation. In some embodiments, the prospective genetic variation comprises a repeat expansion, a mobile element insertion (MEI), or a Uniparental disomy (UPD). In some embodiments, the prospective genetic variation is classified as likely benign, benign, likely pathogenic, pathogenic, or uncertain significance. In some embodiments, a variant classified as likely benign indicates at least about a 90% certainty of benignity. In some embodiments, a variant classified as benign indicates at least about a 99% certainty of benignity. In some embodiments, a variant classified as likely pathogenic indicates at least a 90% certainty of pathogenicity. In some embodiments, a variant classified as pathogenic indicates at least about a 99% certainty of pathogenicity.

In some embodiments, the processing comprises using a trained machine learning algorithm. In some embodiments, the computer processing outputs one or more scores indicative of a likelihood of association of the prospective genetic variation with the phenotype associated with the prospective genetic variation. In some embodiments, the computer processing uses data derived from published literature. In some embodiments, the computer processing uses data derived from Online Mendelian Inheritance in Man, Orphanet, GeneReviews, or a combination thereof. In some embodiments, the computer processing uses data derived from two or more related individuals.

In some embodiments, the set of pre-determined criteria is based at least upon a frequency of the prospective genetic variation in affected and unaffected populations of a disease or disorder. In some embodiments, the set of pre-determined criteria is based at least upon a variant type. In some embodiments, the set of pre-determined criteria is based at least upon a disease mechanism. In some embodiments, the set of pre-determined criteria is based at least upon segregation patterns of one or more loci. In some embodiments, the set of pre-determined criteria is based at least upon data from an individual and a data from a mother or father of the individual. In some embodiments, the set of pre-determined criteria is derived at least in part from analysis of functional studies, case studies, or cohort studies.

In some embodiments, the method is performed in less than 7 days. In some embodiments, the method is performed in less than 10 days. In some embodiments, the method is performed with an accuracy of at least about 90%.

Aspects disclosed herein provide methods for (a) obtaining training information comprising a set of genes and a set of phenotypes, (b) processing the training information to identify a prospective genetic variation and a benign phenotype associated with the prospective genetic variation, wherein the benign phenotype comprises an absence of a disease or disorder, (c) annotating the prospective genetic variation with the benign phenotype to produce an annotated genetic variation, and (d) incorporating the annotated genetic variation into a gene-phenotype knowledge base.

In some embodiments, the training sequencing information comprises information derived from sequencing one or more individuals.

In some embodiments, the method further comprises, prior to processing the training information to identify a prospective genetic variation and a benign phenotype associated with the prospective genetic variation, obtaining data relating to a medical records or clinical notes corresponding to the one or more individuals. In some embodiments, one or more phenotypes of the one or more individuals is identified based at least upon the medical records or the clinical notes.

In some embodiments, the sequencing comprises a whole genome sequencing. In some embodiments, the sequencing comprises an exome sequencing reaction. In some embodiments, the sequencing comprises a targeted sequencing reaction. In some embodiments, the targeted sequencing reaction comprises using one or more pull down probes.

In some embodiments, one or more individuals comprise a fetus or a newborn. In some embodiments, one or more individuals comprise an individual younger than 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, 3 years old, 2 years old, or 1 year old. In some embodiments, one or more individuals comprise an adult.

In some embodiments, the method further comprises, prior to processing the training information to identify a prospective genetic variation and a benign phenotype associated with the prospective genetic variation, the prospective genetic variation was previously classified as a variant of uncertain significance. In some embodiments, the method further comprises, prior to processing the training information to identify a prospective genetic variation and a benign phenotype associated with the prospective genetic variation, the prospective genetic variation was previously classified as likely benign, likely pathogenic, or pathogenic. In some embodiments, a prospective genetic variation classified as likely benign indicates at least about a 90% certainty of benignity. In some embodiments, a genetic variation classified as benign indicates at least a 99% certainty of benignity. In some embodiments, a genetic variation classified as likely pathogenic indicates at least about a 90% certainty of pathogenicity. In some embodiments, a genetic variation classified as pathogenic indicates at least a 99% certainty of pathogenicity.

In some embodiments, the processing comprises using a trained machine learning algorithm. In some embodiments, the processing outputs one or more scores indicative of strength of association of the prospective genetic variation with the phenotype associated with the prospective genetic variation. In some embodiments, the processing uses data derived from published literature. In some embodiments, the processing uses data derived from Online Mendelian Inheritance in Man, Orphanet, GeneReviews, or a combination thereof. In some embodiments, the processing uses data derived from two or more related individuals.

In some embodiments, the set of pre-determined criteria is based at least upon a frequency of the prospective genetic variation in affected and unaffected populations of a disease or disorder. In some embodiments, the set of pre-determined criteria is based at least upon a variant type. In some embodiments, the set of pre-determined criteria is based at least upon a disease mechanism. In some embodiments, the set of pre-determined criteria is based at least upon segregation patterns of one or more loci. In some embodiments, the set of pre-determined criteria is based at least upon data from an individual and a data from a mother or father of the individual. In some embodiments, the set of pre-determined criteria is derived at least in part from analysis of functional studies, case studies, or cohort studies.

In some embodiments, the method is performed in less than 7 days. In some embodiments, the method is performed in less than 10 days. In some embodiments, the method is performed with an accuracy of at least about 90%.

Aspects disclosed herein provide methods for (a) obtaining sequencing information derived from one or more individuals, (b) processing the sequencing information to identify a genetic variation and a phenotype associated with the genetic variation, wherein the method is performed in less than 7 days with an accuracy of at least about 90%, and wherein said accuracy can be determined as a likelihood that said phenotype is truly associated with said genetic variation in said one or more individuals.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of automated variant calling versus vetted variant calling.

FIG. 2 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out.

The term “American College of Medical Genetics and Genomics (ACMG) Criteria”, as used herein, generally refers to a variant classification system that assigns criteria to different types of evidence and is based on the guidelines first described in Richards S, et al. Genet. Med. 2015 (PMID 25741868, which is incorporated by reference herein in its entirety) and later by McCormick EM, et al. Hum Mutator 2020 (PMID 32906214, which is incorporated by reference herein in its entirety) or a customized set of guidelines.

The term “candidate status”, as used herein, generally refers to considered possible/emerging disease-gene associations. These diseases may be referred to as a gene-related disorder.

The term “clinical significance”, as used herein, generally refers to a clinically discernible phenotype of a genetic variation on a subject and/or a clinical effect of a genetic variant on a subject (e.g., an influence on clinical management for the subject). The clinical significance may be affected by functional/biological effects, however, a higher threshold may be required for genetic variation to impart a clinical effect than a functional effect. For example, genetic variation that cause significant functional effects may be likely to cause a clinically discernible phenotype. Conversely, genetic variation with no or a modest functional phenotype may not be expected to have a clinically discernible phenotype.

The term “Clin Var”, as used herein, generally refers to a publicly available archive of human genetic variants and their relationships with health and disease. Variant submissions provide information on the submitter as well as documentation of evidence supporting the ClinVar variant classification as described in Landrum M, et al. Nucleic Acid Res. 2018 (PMID 29165669, which is incorporated by reference herein in its entirety).

The term “disease status”, as used herein, generally refers to the evidence for a gene-disease association. For example, options may include Validated, Candidate, New, and Rejected.

The term “disease status worksheet”, as used herein, generally refers to a scoring worksheet that weighs different types of evidence to determine if a disease association is validated or not. It can be started by genetic counselors, variant scientists, analysts, or gene experts but may be reviewed by human experts.

The term “dominant negative” or “DN”, as used herein, generally refers to a disease mechanism where the altered gene product negatively affects the function of the wild-type product. Dominant negative mechanisms are determined using the GOF checklist and may be approved by human experts.

The term “gain-of-function” or “GOF”, as used herein, generally refers to a disease mechanism where the function of an altered gene product is increased/enhanced (hypermorph) or gains a new, abnormal function (neomorph). GOF mechanisms may be determined using a GOF checklist and may be approved by human experts.

The term “gene expert”, as used herein, generally refers to an individual or group of people that are considered experts on a specific gene or groups of genes associated with a specific phenotype.

The term “gene-phenotype knowledge base”, as used herein, generally refers to a database for storing gene-phenotype (e.g., gene-disease) related content for use in variant interpretation. The database content may be related to functional domains and disease mechanisms. The gene-phenotype knowledge base may comprise a web-based application for storing and curating information for loci (i.e., genes) and phenotypes (e.g., diseases). The gene-phenotype knowledge base may store information from external sources as well as internal curated data to serve as a single source of truth for gene-disease-phenotype information.

The term “genetic variation”, as used herein, generally refers to a variation in a genome of a subject as compared to a reference genome (e.g., generated from one or more reference subjects).

The term “GnomAD”, as used herein, generally refers to the Genome Aggregation Database (gnomAD), originally launched in 2014 as the Exome Aggregation Consortium (ExAC), which is a coalition of investigators seeking to aggregate and harmonize exome and genome sequencing data from a variety of large-scale sequencing projects, and to make summary data available for the wider scientific community as described in Lek M, et al. Nature 2016 (PMID 27535533, which is incorporated by reference herein in its entirety).

The term “HGMD”, as used herein, generally refers to the Human Genetic Mutation Database (HGMD), a database that has a comprehensive list of published variants in association with inherited disease as described in Stenson PD, et al. Hum. Genet. 2014 (PMID: 24077912, which is incorporated by reference herein in its entirety).

The term “locus”, as used herein, generally refers to a particular genomic region. A locus may be associated with a particular disease or disorder (e.g., inherited disorder). A locus may comprise a gene or a portion of a gene, but may also include other types of genomic regions, such as cytogenetic regions.

The term “loss-of-function” or “LOF”, as used herein, generally refers to a disease mechanism that results in the reduction or loss of normal biological function of a gene product. LOF mechanisms may be determined using an LOF worksheet and may be approved by human experts.

The term “mechanism worksheet”, as used herein, generally refers to a scoring worksheet that weighs different types of evidence to determine if a disease mechanism can be established for a specific disease. Mechanism options include loss of function (LOF; amorph), Gain of function (GOF; neomorph, hypermorph), Dominant negative (DN; antimorph) or unknown. Clinical staff and Variant scientists may start the worksheet and it may be approved by human experts.

The term “new status”, as used herein, generally refers to diseases imported into a gene-disease database that have not yet been reviewed by a curator.

The term “non-sense mediated mRNA decay (NMD)”, as used herein, generally refers to a control mechanism that results in selective degradation of mutant protein that is too short, and has the potential to be detrimental to the cell when expressed.

The term “Online System for Clinical Analysis of Results (OSCAR)”, as used herein, generally refers to an information tracking system for laboratory and analysis data.

The term “OMIM”, as used herein, generally refers to the Online Mendelian Inheritance in Man (OMIM), which is an online catalog of human genes and genetic disorders. The OMIM database focuses on the genotype-phenotype relationship between genes and diseases. (Online Mendelian Inheritance in Man, OMIM®. McKusick-Nathans Institute of Genetic Medicine, Johns Hopkins University (Baltimore, MD): omim.org/).

The term “pathogenicity”, as used herein, generally refers to an ability of a genetic variation to cause disease. The pathogenicity of a genetic variation may be indicated by a likelihood or probability that a subject with the genetic variation may be afflicted with the disease. For example, a pathogenicity may be likely benign, benign, likely pathogenic, pathogenic, or uncertain significance.

The term “phenotype”, as used herein, generally refers to a discernible characteristic of a subject, such as appearance, behavior, and development. It can include observable physical traits, biochemical properties, physiological properties, or disease traits (e.g., presence of disease, absence of disease, elevated risk of disease, normal risk of disease, or decreased risk of disease).

The term “rejected status”, as used herein, generally refers to a status selected by a human reviewer if the disease association or functional domain is not real based on refuting data or the functional domain is no longer valid.

The term “validated status”, as used herein, generally refers to gene-disease associations considered to be sufficiently substantiated by published data or functional domains with gene expert approval and sufficient functional evidence supporting curation.

The term “ES”, as used herein, generally refers to exome sequencing.

The term “GS”, as used herein, generally refers to genome sequencing.

The term “SNV”, as used herein, generally refers to single nucleotide variation.

The term “VUS”, as used herein, generally refers to a variant of uncertain significance.

The term “WES”, as used herein, generally refers to whole exome sequencing.

The term “WGS”, as used herein, generally refers to whole genome sequencing.

The majority of rare diseases are serious genetic conditions associated with substantial morbidity and mortality that collectively impact 25 to 30 million people in the United States. These conditions can be challenging to diagnose, often with years-long invasive and costly diagnostic odysseys including the involvement of numerous specialists ordering serial genetic testing and costly diagnostic procedures or interventions.

There are more than 7,000 different diseases that are considered rare. Each condition affects a few hundred to a few thousand people. Collectively, however, they impact about 25 to 30 million people in the United States. About 85% of rare diseases are estimated to be genetic with the majority being serious conditions associated with substantial morbidity and mortality with a considerable medical and financial burden to patients and their families (Tisdale, A., Cutillo, C. M., Nathan, R., Russo, P., Laraway, B., Haendel, M., Nowak, D., Hasche, C., Chan, C. H., Griese, E., Dawkins, H., Shukla, O., Pearce, D. A., Rutter, J. L., & Pariser, A. R. (2021). The IDeaS initiative: pilot study to assess the impact of rare diseases on patients and healthcare systems. Orphanet J Rare Dis, 16(1), 429. doi.org/10.1186/s13023-021-02061-3, which is incorporated by reference herein in its entirety).

These rare diseases can be challenging to diagnose with patients often experiencing years-long diagnostic odysseys, typically involving numerous clinical assessments and investigations that can be invasive and costly (Tan, T. Y., Dillon, O. J., Stark, Z., Schofield, D., Alam, K., Shrestha, R., Chong, B., Phelan, D., Brett, G. R., Creed, E., Jarmolowicz, A., Yap, P., Walsh, M., Downie, L., Amor, D. J., Savarirayan, R., McGillivray, G., Yeung, A., Peters, H., White, . . . S. M. (2017). Diagnostic Impact and Cost-effectiveness of Whole-Exome Sequencing for Ambulant Children With Suspected Monogenic Conditions. JAMA Pediatr, 171(9), 855-862.

doi.org/10.1001/jamapediatrics.2017.1755, which is incorporated by reference herein in its entirety). It has been reported the average time for an accurate diagnosis is approximately 4 to 5 years, but may take over 10 years (Marwaha, S., Knowles, J. W., & Ashley, E. A. (2022). A guide for the diagnosis of rare and undiagnosed disease: beyond the exome. Genome Med, 14(1), 23.
doi.org/10.1186/s13073-022-01026-w, which is incorporated by reference herein in its entirety). The lack of diagnosis or misdiagnoses may result in inappropriate care, lack of targeted treatment, and missed opportunities for interventions that may improve or prevent disease progression (Tisdale et al., 2021).

Establishing an accurate underlying diagnosis based on clinical signs and symptoms is often challenging due to variable presentation and disease course as well as numerous possible genetic causes (Clark, M. M., Stark, Z., Farnaes, L., Tan, T. Y., White, S. M., Dimmock, D., & Kingsmore, S. F. (2018). Meta-analysis of the diagnostic and clinical utility of genome and exome sequencing and chromosomal microarray in children with suspected genetic diseases. NPJ Genom Med, 3, 16. doi.org/10.1038/s41525-018-0053-8, which is incorporated by reference herein in its entirety; Malinowski, J., Miller, D. T., Demmer, L., Gannon, J., Pereira, E. M., Schroeder, M. C., Scheuner, M. T., Tsai, A. C., Hickey, S. E., & Shen, J. (2020). Systematic evidence-based review: outcomes from exome and genome sequencing for pediatric patients with congenital anomalies or intellectual disability. Genet Med, 22(6), 986-1004. doi.org/10.1038/s41436-020-0771-z, which is incorporated by reference herein in its entirety). Current methods of genetic testing may be performed serially, using chromosome microarray, single gene analysis, and multi-gene panels, contributing to diagnostic delays or no diagnosis. This approach relies on the subjective assessment of clinicians who may have never encountered a patient with the same constellation of findings, or the findings may be non-specific making a differential diagnosis difficult.

Recognizing the above needs, the present disclosure provides methods and systems that address the needs of patients diagnosed with rare disorders and the clinicians treating these conditions. Methods and systems of the present disclosure may comprise genome sequencing and/or exome sequencing for pediatric, rare, and ultra-rare diseases. These methods and systems may comprise delivering personalized, actionable insights that improve health outcomes.

Methods and systems of the present disclosure may comprise sequencing a large number (e.g., >700,000) of clinical genomes and exomes and obtaining a large number (e.g., >5.2 million) of expertly annotated phenotypes. Combined, this has resulted in one of the largest clinical rare-disease datasets, leading to more accurate result interpretation, and minimized uncertainty (Richards et al., 2015). Sequencing coverage may be optimized for uniformity and depth of coverage, and processes (e.g., polymerases, capture reactions) may comprise improvements in sequencing fidelity for capturing and analyzing genomic anomalies such as indels, de novo events, copy number variant (CNV) detection, GC-rich regions, among others. Methods and systems of the present disclosure may encompass knowledge that includes integration of evidence from unparalleled internal database and variant classification according to most recent guidelines (Landrum, M. J., & Kattman, B. L. (2018). ClinVar at five years: Delivering on the promise. Hum Mutat, 39(11), 1623-1630. doi.org/10.1002/humu.23641, which is incorporated by reference herein in its entirety; Richards, S., Aziz, N., Bale, S., Bick, D., Das, S., Gastier-Foster, J., Grody, W. W., Hegde, M., Lyon, E., Spector, E., Voelkerding, K., & Rehm, H. L. (2015). Standards and guidelines for the interpretation of sequence variants: a joint consensus recommendation of the American College of Medical Genetics and Genomics and the Association for Molecular Pathology. Genet Med, 17(5), 405-424. doi.org/10.1038/gim.2015.30, which is incorporated by reference herein in its entirety).

Genetics experts and genetic counselors may develop improvements with appropriate test selection, clinical questions, result interpretation, patient support, pretest genetic education and test coordination, and post-test genetic counseling. Additionally, test utilization management is performed to ensure genetic test orders are clinically appropriate and leads to a reduction in healthcare costs by preventing duplicate, incorrect, or unnecessary testing.

### Genetic Variations

Data (e.g., sequencing data) may be evaluated for the presence of genetic variation. The sequencing data may be mapped or aligned with a reference genome to identify genetic variations from a reference genome. The genetic variation may be mapped to a specific gene or loci and may be annotated to indicate that the genetic variation is present at a specific gene or loci. The genetic variations may be annotated to indicate the type of genetic variation. For example, the genetic variation may be mapped to a specific gene and identified as an insertion, deletion, transposition, single nucleotide variation, copy number variation, a fusion gene, or other variation. The genetic variation may comprise a repeat expansion, a mobile element insertion (MEI) or a Uniparental disomy (UPD). The genetic variations may be annotated based at least in part on zygosity. For example, genetic variation may be present on one allele of a subject (e.g., heterozygous or hemizygous) or both alleles of a subject (e.g., homozygous). The genetic variation may be organized into dataset that is annotated based on various features of the genetic variation.

Genetic variations may be annotated based on clinical data or sequencing data from other subjects. For example, multiple related subjects may be sequenced and analyzed. The presence of genetic variation in related individuals may allow for the determination of an inheritance pattern or segregation pattern. The genetic variations may be annotated to include the inheritance pattern or segregation pattern.

Genetic variations may be annotated based on gene ontology or molecular relationships with other genes. For example, a genetic variation may be present in a gene that interacts with or has a previously known relationship with another gene. The genetic variation may be annotated such to indicate that it is present in a given gene and has a relationship to the other gene. For example, the gene may be relevant for a known cellular function (e.g., as a part of a cell signaling pathway), or previously associated with a condition (e.g., a gene that is a known cancer biomarker). For example, the data may be annotated relating to protein-protein interactions, gene family data, orthologs, paralogs, biological pathways, gene-phenotype data from animal sources, or combinations thereof.

The genetic variations may be annotated based on a pathogenicity or benignity for a condition or disorder. For example, the genetic variation may be pathogenic, likely pathogenic, likely benign, benign, or uncertain significance. The genetic variation may be a variation of uncertain significance (VUS). The genetic variation may be initially annotated as a VUS and may be re-annotated, for example, based on methods provided in this disclosure, to another category. For example, a variation may be annotated as VUS and then re-annotated to be a benign variation.

Similarly, a variation may be initially annotated as a pathogenic, likely pathogenic, likely benign, benign variation, or variant of uncertain significance, and may be reannotated, for example, based on methods provided in this disclosure, to be another of pathogenic, likely pathogenic, likely benign, benign, or uncertain significance.

Annotated genetic variations may be grouped in a dataset that may be searched or filtered. For example, the dataset may be searched to identify all variants that have a particular inheritance pattern. In another example, the genetic variations may be searched as to those associated with a particular cellular function. For example, the data may be searched as to those relating to protein-protein interactions, gene family data, orthologs, paralogs, biological pathways, gene-phenotype data from animal sources, or a combination thereof.

The searchable dataset of genetic variations may allow for a user or computer algorithm to identify a genetic variation as associated to a phenotype. For example, a subject may display a plurality of phenotypic characteristic that is associated with a disorder or condition. Each of the plurality of phenotypic characteristic may be searched in the dataset and a list of genetic variation that match the phenotypic characteristic. Genetic variations that are relevant for multiple of the subjects phenotypic characteristic may be flagged as potentially relevant as associated with the disorder or condition.

### Databases

In some cases, the methods and systems of the present disclosure can utilize data such as, for example, genomic data, phenotypic data, exomic data, proteomic data, metabolomic data, microbiomics data, additional data, portions thereof, or any combination thereof. Examples of genomic data include, but are not limited to, whole genome sequencing data, single nucleotide polymorphism data, mutations, insertions, deletions, copy number variation data, gene expression data, genomic structural variant data, or the like, or any combination thereof. Examples of phenotypic data include, but are not limited to, physical features of the subject (e.g., height, weight, blood type, eye color, age, etc.), or the like, or any combination thereof. Examples of exomic data includes, but are not limited to, sequencing data from a protein encoding genomic region, insertions, deletions, splice variants, variations, or the like, or any combination thereof. Examples of proteomic data include, but are not limited to, protein sequences, protein structures, protein-protein interactions, post-translational modifications, protein expression levels, or the like, or any combination thereof. Examples of metabolomic data include, but are not limited to, metabolite concentrations, metabolic pathways, metabolite-metabolite interactions, enzyme activity data, metabolite structure, or the like, or any combination thereof. Examples of microbiomics data include, but are not limited to, microbiomics taxonomy (e.g., an identity of the organisms of the microbiome), microbiomics abundance (e.g., a number of organisms), microbiomics gene sequence or expression data, microbe-microbe interactions, or the like, or any combination thereof. Examples of additional data include, but are not limited to, clinical data, medical histories, diagnostic test results, imaging data, physical examination data, trail participation, clinical notes, or the like, or any combination thereof.

In some cases, the data can be sourced from one or more sources. For example, the data can be sourced from a plurality of sources. Examples of sources include, but are not limited to, trials (e.g., clinical trials, etc.), subjects (e.g., the subject can provide the data), clearinghouses, medical records, clinical notes (e.g., summarizing interactions that occur between a patient and a healthcare provider, such as ICD-10 codes), surveys, registries, user data (e.g., user data from a mobile application, etc.), pharmacy records, databases (e.g., public databases, private databases, etc.), or the like, or any combination thereof. In some cases, the subjects can be identified as candidates for the methods and/or systems of the present disclosure by, for example, using the data associated with the subject. For example, a subject's medical records can be used to identify the subject as being a good candidate for analysis by the methods of the present disclosure. In another example, trial data can be received and candidates from the trial data can be identified. In some cases, the data can be generated specifically for use in the methods and systems of the present disclosure. For example, the data can be generated de novo for use in the methods of the present disclosure. In some cases, the data can be gathered from other sources (e.g., sources not specific to the methods of the present disclosure). For example, the data can be generated and then accessed for use with the methods and systems of the present disclosure.

In various aspects, one or more databases are generated. The one or more databases may comprise data on one or more genes. The databases may comprise data relating to genes and associated diseases. The one or more databases may comprise data relating to a set of genes and a set of gene-phenotype associations (GPAs) associated with the set of genes. The one or more databases may comprise data derived from Online Mendelian Inheritance in Man (OMIM), Orphanet, GeneReviews, or a combination thereof. The one or more databases may comprise Human Phenotype Ontology (HPO) terms. The one or more databases may comprise data relating to disease mechanisms or causative association of genes and diseases. The one or more databases may comprise data relating the gene function. The more databases may comprise data relating to phenotypes associated with genes or genetic variations in a given gene. The databases may comprise descriptions of phenotypes. For example, the description may comprise patient descriptions, clinical observations, or other clinical data.

The one or more databases may comprise data relating to population genomics. The one or more databases may comprise data relating to frequency of populations that are affected (e.g., an allele is present) or unaffected (e.g., an allele is absent) by a given allele or genetic variation. The one or more databases relating to population genomics may be used to analyze the presence of a pathogenic or likely pathogenic genetic variation in a population. The one or more databases may allow for individuals with rare or less documented genetic variations to be more easily identified.

The one or more databases may comprise data derived from mining or analyzing published literature (e.g., using a literature surveyor). The literature mining may comprise automated retrieval and formatting of text documents to construct a comprehensive corpus. In some embodiments, the literature mining may comprise processing the comprehensive corpus using natural language processing (NLP) to identify GPAs without manual discovery. In some embodiments, the text documents may be retrieved from one or more article databases. In some embodiments, the article databases may comprise MEDLINE/Pubmed, PMC articles from NCBI, GeneReviews, or a combination thereof. In some embodiments, the literature mining may comprise generating vector embeddings from the text documents in the comprehensive corpus. In some embodiments, the vector embeddings may be generated using a trained doc2vec model. In some embodiments, the literature mining may comprise training a doc2vec model configured to learn and generate vector embeddings from the text documents in the comprehensive corpus. In some embodiments, (a) may comprise computer processing Human Phenotype Ontology (HPO) terms. In some embodiments, (a) may comprise computer processing non-exact HPO terms. In some embodiments, (a) may comprise determining semantic similarity between HPO terms and/or non-exact HPO terms.

### Data Processing

In various aspects, one or more database comprising gene-phenotype knowledge may be used to identify prospective genetic variations and a phenotype associated with the prospective genetic variation. Data from a subject (e.g., sequencing data or clinical data relating to phenotype) may be obtained for a subject. The gene-phenotype knowledge base may be used to generate a plurality of scores, based at least in part on the GPAs associated with the set of genes. The scores may be indicative of a likelihood of association subject's phenotype and a gene (or genes) of the set of genes. The scores may comprise a hybrid relative semantic similarity (HRSS) score, a Jaccard similarity score, a word2vec cosine similarity score, a doc2vec score, or a combination thereof.

In various aspects, computer processing may be performed on one or more database described throughout this disclosure (e.g., a database comprising gene-phenotype knowledge) to produce an output. The computer processing may be performed using one or more input databases or input datasets. For example, the input dataset may comprise one or more of: disease-phenotype annotations, patient descriptions, and structured clinical data. In some embodiments, the input dataset may comprise two or more of: disease-phenotype annotations, patient descriptions, and structured clinical data. In some embodiments, the input dataset may comprise disease-phenotype annotations, patient descriptions, and structured clinical data. In some embodiments, the one or more databases may comprise Online Mendelian Inheritance in Man (OMIM), Orphanet, GeneReviews, or a combination thereof.

The computer processing may use a trained machine learning model. The trained machine learning model may be trained on one or more of the databases as described throughout this disclosure. For example, the trained machine learning model may by trained on Online Mendelian Inheritance in Man (OMIM), data derived from the literature mining, data relating to inheritance patterns, or protein-protein interactions, or other data. In some embodiments, the trained machine learning model may be selected from the group consisting of a random forest, a neural network, a decision tree, a k-nearest neighbor, a linear regression, a logistic regression, and a combination thereof

In various embodiments, the processing (e.g., processing with a trained algorithm) may output a predicted likelihood of association of a gene variation to a phenotype. In various embodiments, the processing (e.g., processing with a trained algorithm) may output a predicted pathogenicity of a genetic variation.

In various aspects, a set of pre-determined criteria is applied to using a human interpretation. The pre-determined criteria may be applied for a human determination of a likelihood of an association of a phenotype and a genetic variation. The pre-determined criteria may be applied for a human determination of a clinical significance of a phenotype and a genetic variation. For example, a trained algorithm may provide an assessment that a genetic variation and a phenotype are associated. A human, using pre-determined criteria may confirm the assessment of the trained algorithm. Similarly, a human, using pre-determined criteria may reject the assessment of the trained algorithm.

The pre-determined criteria may be based at least upon a frequency of the genetic variation in affected and unaffected populations for a disease or disorder. The pre-determined criteria may be based at least upon a type of genetic variation. The pre-determined criteria may be based at least upon a disease mechanism. The set of pre-determined criteria may be based at least upon segregation patterns of one or more loci. The set of pre-determined criteria is derived at least in part from analysis of functional studies, case studies, or cohort studies. The set of pre-determined criteria may be based at least upon data from the subject and optionally a data from a mother or father of the subject. For example, the set of pre-determined criteria may be based at least upon data from the subject without data from a mother or father of the subject (e.g., proband-only data). As another example, the set of pre-determined criteria may be based at least upon data from the subject with data from a mother or father of the subject. For example, the data from a mother or father of the subject may indicate the inheritance of the genetic variation or if the variation is a de novo genetic variation. The set of pre-determined criteria may be based at least on the location of a variation within a given gene. For example, the genetic variation may be present in a functional domain of a protein.

The systems and method of the disclosure may be performed with a certain accuracy. For example, the system and methods may comprise an accuracy at determining an association of a phenotype and a genetic variation. For example, the system and methods may comprise an accuracy at determining the pathogenicity of a genetic variation. The systems and methods of the disclosure may be performed at an accuracy of at least about 60%, at least about 70%, at least about 75%, at least about 80%, at least about 85%, at least about 90%, at least about 95%, at least about 98%, or at least about 99%.

The systems and method of the disclosure may be performed and completed within a certain time frame. For example, the system and methods may determine an association of a phenotype and a genetic variation within a certain time frame. For example, the system and methods may determine the pathogenicity of a genetic variation within a certain time frame. The systems and methods of the disclosure may be performed and completed in less than about 20 days, less than about 15 days, less than about 10 days, less than about 7 days, or less than about 5 days.

### Samples

The methods and/or systems of the present disclosure may be suitable for analyzing a variety of biomolecules (e.g., nucleic acid molecules, proteins, polypeptides, carbohydrates, fragments thereof, etc.) derived from any of a variety of samples and/or sources. The nucleic acid molecules may be deoxyribonucleic acid, ribonucleic acid, etc. In some cases, the nucleic acid molecules may be derivatives of one or more nucleic acid (e.g., compliments, reverse compliments, fragments, or the like).

The methods and/or systems of the present disclosure may use a variety of sequencing and/or imaging applications and for sequencing and/or identifying biomolecules (e.g., nucleic acid molecules) derived from any of a variety of samples and sources. Biomolecules (e.g., nucleic acids), in some cases, may be extracted from any of a variety of biological samples, e.g., blood samples, saliva samples, urine samples, cell samples, tissue samples, and the like. The samples of the present disclosure may be extracted from one or more sources. For example, a tissue sample can be extracted from a subject. The samples of the present disclosure may be extracted from other samples. For example, a plurality of cells can be extracted from a tissue. In another example, a biomolecule (e.g., a nucleic acid molecule) can be extracted from a cell.

For example, the disclosed devices and systems may be used for the analysis of biomolecules (e.g., nucleic acids molecules) derived from any of a variety of different cell, tissue, or sample types. For example, nucleic acids may be extracted from cells, or tissue samples comprising one or more types of cells, derived from eukaryotes (such as animals, plants, fungi, protista), archaebacteria, or eubacteria. In some cases, biomolecules (e.g., nucleic acids) may be extracted from prokaryotic or eukaryotic cells, such as adherent or non-adherent eukaryotic cells. Biomolecules (e.g., nucleic acids) can be variously extracted from, for example, primary or immortalized rodent, porcine, feline, canine, bovine, equine, primate, or human cell lines. Biomolecules (e.g., nucleic acids) may be extracted from any of a variety of different cell, organ, or tissue types (e.g., white blood cells, red blood cells, platelets, epithelial cells, endothelial cells, neurons, glial cells, astrocytes, fibroblasts, skeletal muscle cells, smooth muscle cells, gametes, or cells from the heart, lungs, brain, liver, kidney, spleen, pancreas, thymus, bladder, stomach, colon, cheek (e.g., buccal cells), or small intestine). Biomolecules (e.g., nucleic acids) may be extracted from normal or healthy cells. Alternately or in combination, the biomolecules can be extracted from diseased cells, such as cancerous cells, or from pathogenic cells that are infecting a host. Some nucleic acids may be extracted from a distinct subset of cell types, e.g., immune cells (such as T cells, cytotoxic (killer) T cells, helper T cells, alpha beta T cells, gamma delta T cells, T cell progenitors, B cells, B-cell progenitors, lymphoid stem cells, myeloid progenitor cells, lymphocytes, granulocytes, Natural Killer cells, plasma cells, memory cells, neutrophils, eosinophils, basophils, mast cells, monocytes, dendritic cells, and/or macrophages, or any combination thereof), undifferentiated human stem cells, human stem cells that have been induced to differentiate, rare cells (e.g., circulating tumor cells (CTCs), circulating epithelial cells, circulating endothelial cells, circulating endometrial cells, bone marrow cells, progenitor cells, foam cells, mesenchymal cells, or trophoblasts). The biomolecules (e.g., nucleic acids) may optionally be attached to one or more moieties (e.g., non-nucleotide moieties) such as labels and other small molecules, large molecules (such as proteins, lipids, sugars, etc.), and/or solid or semi-solid supports, for example through covalent or non-covalent linkages with either end of a biomolecule (e.g., the 5′ or 3′ end of the nucleic acid). Labels can include any moiety that is detectable using any of a variety of detection methods known to those of skill in the art, and thus renders the attached oligonucleotide or nucleic acid similarly detectable. Some labels, e.g., fluorophores, can emit electromagnetic radiation that is optically detectable or visible. The labels may comprise one or more barcode molecules (e.g., molecules configured to provide a detectable signal corresponding to an identity of the molecule).

The one or more samples may comprise one or more label moieties. Examples of label moieties include, but are not limited to, optical barcodes, nanoparticles, magnetic moieties, or electrical moieties, or any combination thereof. For example, the one or more samples may comprise a plurality of barcode tags. For example, the one or more samples can be treated with a plurality of barcode tags configured to bind to a plurality of analytes (e.g., nucleic acids, proteins, carbohydrates, etc.) contained within the one or more samples.

### Subjects

In some cases, the methods and/or systems of the present disclosure may be applied to data related to one or more subjects. The one or more subjects may be human subjects. For example, the data analyzed may be data associated with human subjects. In some cases, the human subjects may be an infant (e.g., under 1 year of age). In some cases, the human subject may be a child (e.g., under 18 years of age). In some cases, the human subject may be an adult (e.g., over 18 years of age). For example, the data analyzed by the methods and systems of the present disclosure can be associated with a plurality of subjects each under 1 year old. In another example, the data analyzed by the methods and systems of the present disclosure can be associated with a plurality of subjects of any ages (e.g., a mixed cohort).

In some cases, the data analyzed by the methods and systems of the present disclosure can be taken from a cohort (e.g., plurality) of subjects. The cohort may comprise at least about 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 110,000, 120,000, 130,000, 140,000, 150,000, 160,000, 170,000, 180,000, 190,000, 200,000, 210,000, 220,000, 230,000, 240,000, 250,000, or more subjects. The cohort may comprise at most about 250,000, 240,000, 230,000, 220,000, 210,000, 200,000, 190,000, 180,000, 170,000, 160,000, 150,000, 140,000, 130,000, 120,000, 110,000, 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, or less subjects. The cohort may comprise a number of subjects in a range as defined by any two of the preceding values.

### Sequencing

In some cases, data used in the present disclosure may be sequencing data (e.g., genomic data, exomic data, etc.). The sequencing data may be generated by sequencing one or more nucleic acids from a sample from a subject. The sequencing may comprise sequencing via sequencing-by-synthesis, sequencing-by-ligation, Sanger sequencing, hydrogen ion detection sequencing, polony sequencing, nanopore sequencing, rolling circle sequencing, pyrosequencing, or the like, or any combination thereof. The sequencing may be performed on nucleic acids derived from samples as described elsewhere herein. In some cases, the sequencing may comprise protein sequencing (e.g., Edman degradation, mass spectrometry, nuclear magnetic resonance spectroscopy, x-ray crystallography, etc.).

The sequencing may comprise use of a panel. The panel may comprise a targeted sequencing of a plurality of genes or genetic regions of a nucleic acid. The panel may comprise at least about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or more genes or genetic regions. The panel may comprise at most about 10000, 9000, 8000, 7000, 6000, 5000, 4000, 3000, 2000, 1000, 900, 800, 700, 600, 500, 400, 300, 200, 100, 90, 80, 70, 60, 50, 40, 30, 20, 10, or fewer genes or genetic regions. The panel may comprise a number of genes or genetic regions in a range as defined by any two of the preceding values.

Prior to the sequencing, a sample may undergo one or more preparation operations. Examples of preparation operations include, but are not limited to, ligation, amplification, fragmentation, end repair, normalization, or the like, or any combination thereof. For example, a buccal cell sample can be taken from a subject, the nucleic acid molecules of the sample can be extracted, amplified, and sequenced. In this way, data related to the subject can be generated and used in the methods and systems of the present disclosure.

The sequencing may comprise use of one or more pulldown probes. The one or more pulldown probes may be configured to selectively isolate and/or enrich one or more nucleic acid sequences. For example, introduction of the one or more pulldown probes can be used to enrich a particular gene for analysis from the sample. In some cases, one or more orthogonal assays (e.g., quantification of another analyte to confirm a determination provided by the sequencing). For example, a chromosomal microarray analysis may be performed in parallel to the sequencing. In another example, a multiplex ligation dependent probe amplification may be performed. In some cases, the orthogonal assay may be a confirmatory assay. In some cases, an additional sequencing operation may provide the confirmatory assay.

In various aspects, sequencing data is obtained (e.g., via one or more sequencing assays). The sequencing data may be processed or evaluated. For example, the sequencing data may be analyzed using one or more quality metrics. The quality metric may be related to the accuracy of a read. For example, a given sequencing read may be outputted with a quality score. The quality score may be indicative of a signal or noise of data for a given base in the read. Reads that fall below a specific score may be filtered out and removed from downstream data processing. In some cases the sequencing data may be analyzed for contamination. For example, the sequencing data may be derived from a subject. The sequencing data may be analyzed for one or more reads that are derived from a different source (e.g., of a different subject, specimen, or species). The sequencing reads may be aligned to a reference genome of a different species (e.g., a microbial species) and the read may be identified as belonging to a different species (e.g., a human species or a microbial species). The presence of a read derived from another source may indicate the presence of a contamination. Similarly, the subject may comprise one or more previously known alleles. The sequencing data may be analyzed for alleles that differ from the one or more previously known alleles. The presence of a foreign allele may indicate the presence of a sample swap or a contamination.

### Computer Systems

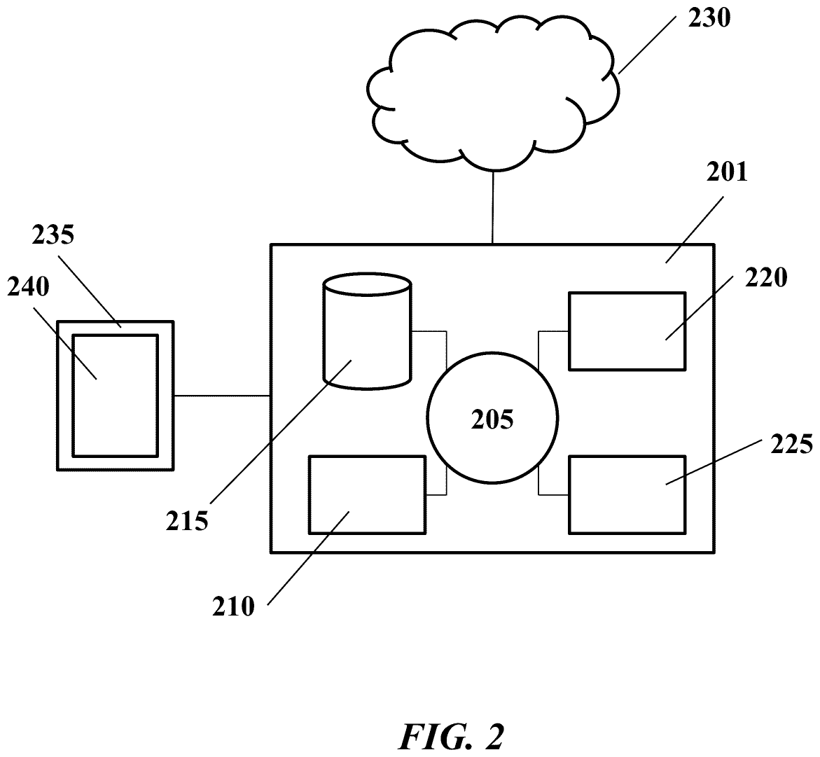

The present disclosure provides computer systems that are programmed to implement methods of the disclosure. FIG. 2 shows a computer system 201 that is programmed or otherwise configured to predict phenotypes from genes. The computer system 201 can regulate various aspects of the present disclosure, such as, for example, gene prioritization or surveying literature. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system.

Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, information about gene prioritization. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, identify genes most likely to be responsible for a clinical presentation.

### EXAMPLES

### Example 1: Gene-Disease Database Curation

Using methods and systems of the present disclosure, gene-disease related content is stored in a gene-disease database for use in variant interpretation. The database content may be related to functional domains and disease mechanisms.

The gene-disease database may comprise a web-based application for storing and curating information for loci (i.e., genes), diseases, and phenotypes. The gene-disease database stores information from external sources as well as internal curated data to serve as the single source of truth for gene-disease-phenotype information. Other internal systems (e.g., OSCAR) use and display gene information for users. The curated information from the gene-disease database is used for variant interpretation. Some human experts perform a curator level role and actively add or edit specific gene-disease information.

The gene-disease database comprises a loci page that stores information about genomic regions (e.g., genes and cytogenic regions). The gene-disease database uses identifiers for genes based on the HUGO Gene Nomenclature Committee (HGNC) ID. Users can search for different loci by gene symbol, full gene name, previous symbols, locus type, location, and tags. Users can sort the list of loci by each of these column headings.

The gene-disease database comprises disease pages that store information about a clinical disorder linked to a locus. Users can search for diseases by name, locus symbol, inheritance, mechanism, source, tags or status. Diseases specific to a locus are listed in the disease table on the loci page.

The diseases table lists all the diseases with known (status=validated) or suspected (status=candidate or new) association with the locus. For each disease association, the table provides information for the disease name, locus name, mode of inheritance, disease mechanism, mutation spectrum, data source, modifiers, tags and status. Human analysts and curators have the ability to sort diseases by multiple different filters (e.g., inheritance, mechanism, etc.). Clinical staff members and curators are able to edit the information in the disease table.

Diseases added to the gene-disease database may default to “unknown” as the disease mechanism. When a disease has been recently validated and/or new functional data is available, human curators evaluate the available evidence to determine if a disease mechanism can be updated from “unknown”.

Disease mechanisms can be curated as Loss-of-function (LOF-Amorph), Gain-of-function (GOF-Neomorph/Hypermorph), Dominant Negative (DN-Antimorph) or Unknown, based on current published variants, internal data, and functional studies. Human curators use mechanism worksheets and checklists to determine the most appropriate disease mechanism based on available evidence. In the event there is insufficient evidence, either no evidence or not enough evidence for LOF/GOF/DN, the disease mechanism remains “unknown”.

Type 1 LOF variants may be a known part of a mutation spectrum. Type 1 LOF variants may use evidence such as published as well as internal LOF variants known to be associated with a specific disease. LOF variants include nonsense, frameshift (excluding protein extension), canonical splice, Met1? And multi-exon deletions in the gene.

Type 2 LOF variants may be a predicted part of a mutation spectrum. Type 2 LOF variants may use evidence such as published functional evidence, animal models, multigene deletions, inheritance and internal LOF variants known to be associated with a specific disease.

Type 3 LOF variants may be a probable part of a mutation spectrum. Type 3 LOF variants may use evidence such as the probability a gene is tolerant to LOF variation (pLI), published as well as internal variants known to be associated with a specific disease.

A GOF checklist is used for diseases with functional studies supporting a GOF effect (hypermorph/neomorph), when there are no LOF variants published in association with the disease or most variants associated with the disease are missense, NMD=No or non-canonical splice variants. Curators must use the approved GOF checklist in Medialab (Gain-of-function (GOF) Checklist).

The GOF checklist may also be used to curate Dominant negative as the disease mechanism. The decision to curate as GOF or Dominant Negative is dependent on the functional evidence available for either mechanism.

The gene-disease database comprises a groups page that stores information about groups of loci, diseases, panels, etc. The groups table on each loci page displays groups that include the locus. The groups table on the loci page lists all relevant groups that gene belongs to, such as, panels the gene is evaluated on or protein families. The group name and status (e.g., validated or new) is listed in the table. Groups are user defined and clinical staff members can add or edit groups.

The gene-disease database comprises a transcripts table that lists all of the current Refseq transcripts associated with the locus. Transcripts can be stored using different filters (e.g., transcript accession, is primary, etc.). Primary transcripts for a gene are designated by a checkmark in the “Is primary transcript” column.

The gene-disease database comprises a domains table that contains relevant domain information for a specific locus and includes curated functional domains, as well as hotspot and coldspot domains. For example, human analysts may assign the appropriate strength for the domain that aligns with the appropriate strength of the applicable ACMG criteria. Domains may be defined by the chromosomal (genomic) coordinates in the gene-disease database. Chromosomal coordinates may be converted to coding and protein positions once a transcript is assigned. Domain citations (references) can be added by PMID or using a unique identifier (non-PMID) in OSCAR. Human analysts can select citations to be added to a particular domain.

The gene-disease database comprises a publications page that contains all publications currently stored in OSCAR publications as well as any new publications that are added within the gene-disease database. Publications can be searched by ID, title, journal name, author and publication date. An “OSCAR” link takes the user to the OSCAR publications page where expert notes, publication PDFs or supplemental material may be found.

The gene-disease database comprises a documents page that contains relevant documents for GeneMB including FAQs and curation worksheets.

The gene-disease database comprises a report section, which provides a gene summary that describes the known gene function, diseases and phenotypes associated with the locus.

### Example 2: Genome Sequencing in an Outpatient Setting

The majority of rare diseases are serious genetic conditions associated with substantial morbidity and mortality that collectively impact 25 to 30 million people in the United States. These conditions can be challenging to diagnose, often with years-long invasive and costly diagnostic odysseys including the involvement of numerous specialists ordering serial genetic testing and costly medical interventions. Genome sequencing (GS) may be used as a single genetic test providing a timely diagnosis to inform appropriate care.

GS provides a broad genetic assessment by targeting both the protein-coding and non-coding regions of the human genome. GS is more comprehensive than ES, as it provides detection of a broad range of variant types and has better or added coverage of certain regions of the genome. Prominent professional societies including the American College of Genetics and Genomics (ACMG) have published evidence-based guidelines strongly recommending GS as a first-tier test. Health economic studies also demonstrate the advantages of GS, with ACMG guidelines stating “compared with standard genetic testing, GS . . . has a higher diagnostic yield and may be more cost-effective when ordered early in the diagnostic evaluation.” GS has a diagnostic rate two to three times higher than traditional genetic testing with a diagnostic yield of ˜40% to 45%. About 60% of patients with a positive GS result may have a change in medical management. These modifications included change in medication (new treatment or halting an existing one), alteration to diet, change in planned procedures or surveillance (surgery, imaging, and/or diagnostic studies), referral to specialist, testing of family members, and/or impact on future reproductive planning. Generally, ample evidence supports GS for patients with a suspected rare genetic disease in the outpatient setting.

There are more than 7,000 different diseases that are considered rare. Each condition affects a few hundred to a few thousand people. Collectively, however, they impact about 25 to 30 million people in the United States. About 85% of rare diseases are estimated to be genetic with the majority being serious conditions associated with substantial morbidity and mortality with a considerable medical and financial burden to patients and their families (Tisdale et al., 2021).

These rare diseases can be challenging to diagnose with patients often experiencing years-long diagnostic odysseys, typically involving numerous clinical assessments and investigations that can be invasive and costly (Tan et al., 2017). It has been reported the average time for an accurate diagnosis is approximately 4 to 5 years, but may take over 10 years (Marwaha et al., 2022). The lack of diagnosis or misdiagnoses may result in inappropriate care, lack of targeted treatment, and missed opportunities for interventions that may improve or prevent disease progression (Tisdale et al., 2021).

Establishing an accurate underlying diagnosis based on clinical signs and symptoms is often challenging due to variable presentation and disease course as well as numerous possible genetic causes (Clark et al., 2018; Malinowski et al., 2020). Traditionally, genetic testing was performed serially, using chromosome microarray, single gene analysis, and more recently multi-gene panels, contributing to diagnostic delays or no diagnosis. This approach relies on the subjective assessment of clinicians who may have never encountered a patient with the same constellation of findings, or the findings may be non-specific making a differential diagnosis difficult. However, in patients with a suspected genetic disease this approach is being replaced with a broad analysis by a single genetic test, by exome sequencing (ES) or genome sequencing (GS) (Clark et al., 2018).

GS, also known as whole genome sequencing (WGS), provides a broad assessment by targeting both the protein-coding and non-coding regions of the human genome (Belkadi et al., 2015; Lelieveld et al., 2015). GS is more comprehensive than ES as it provides (Austin-Tse et al., 2022) detection of a broad range of variant types in 1 test including single nucleotide variants, small insertions and deletions, mitochondrial variants, repeat expansions, copy number variants, and other structural variants broader coverage with better coverage of the exon regions and added coverage in the non-coding regions which include promoter, intronic, and untranslated regions.

GS may be intended for individuals with a suspected rare genetic disorder in which the clinical findings may include congenital anomalies, neurodevelopmental disorders (e.g., epilepsy, developmental delay, intellectual disability, autism spectrum disorders, developmental regression), or dysmorphic features (Clark et al., 2018). However, there are additional clinical findings in which GS is indicated as the symptoms of rare disorders are varied and can impact many body systems which may include hearing, vision, musculoskeletal, skeletal, renal, central nervous, and cardiovascular (Lee, 2023; Sullivan et al., 2023).

Clinical utility studies have reported approximately a 60% change in medical management for individuals with a positive GS result with the rate for GS higher than ES in a recent meta-analysis (Chung et al., 2023). In a systematic evidence review of GS for patients with congenital anomalies (Cas), developmental delay (DD), and/or intellectual disability (ID) initiated by ACMG, 95% of the 167 included studies reported a change to clinical management (Malinowski et al., 2020). These modifications included change in medication (new treatment or halting an existing one), alteration to diet, change in planned procedures or surveillance (surgery, imaging, and/or diagnostic studies), referral to specialist, testing of family members, and/or impact on future reproductive planning (Malinowski et al., 2020). Some of these changes included discontinuation of unnecessary procedures (e.g. diagnostic tissue biopsy), avoidance of certain drugs, or withdrawal of care/start of palliative care (Malinowski et al., 2020).

The standard diagnostic work-up for patients with suspected rare genetic disorders may include radiographs, biopsies, biochemical testing, and serial genetic testing (e.g. chromosomal microarray, single gene testing, multi-gene panels) (Clark et al., 2018; Lavelle et al., 2022). This diagnostic approach is typically a time-consuming and expensive process (Tan et al., 2017). A recent cost-effectiveness analysis from a United States health sector perspective demonstrated GS has a higher detection rate and shortens the diagnostic odyssey, but at a similar cost compared to standard care for children with suspected rare genetic diseases (Incerti et al., 2022). An evidence-based guideline by American College of Medical Genetics and Genomics (ACMG) stated “ . . . GS has a higher diagnostic yield and may be more cost-effective when ordered early in the diagnostic evaluation” (Manickam et al., 2021).

The analytic validation of GS has been published demonstrating a >99% concordance with reference samples (Linderman et al., 2014). In our validation studies, the GS test demonstrated a 100% concordance with previous identified variants in internal samples and >99% sensitivity of variant calls compared with a commonly available reference data set (Genome in a Bottle) (Krusche et al., 2019).

Classes of clinically relevant genetic variation detectable by GS include single nucleotide variations (SNVs); small deletions, duplications, and insertions, repeat expansions, copy number variants, and other structural variants. Coverage may vary by laboratory. At GeneDx, GS coverage is typically 30-40×, which allows detection of additional clinically-significant findings such as copy number variants.

GS has a diagnostic yield two to three times higher than traditional genetic testing (e.g. chromosomal microarray, single gene or targeted panel testing) (Clark et al., 2018; Health Quality Ontario, 2020; Incerti et al., 2022). Also, in patients with negative ES, GS has identified a diagnosis in additional 10-15% of cases (Bertoli-Avella et al., 2021; Lionel et al., 2018; Splinter et al., 2018; Sullivan et al., 2023).

The diagnostic yield of GS is ˜40% as reported by a systematic review and meta-analysis of 37 studies, which included >20,000 patients with suspected rare disease (Clark et al., 2018). A technology assessment that included 9 studies with unexplained development disability and/or multiple congenital anomalies also reported about a 40% diagnostic yield for GS (Health Quality Ontario, 2020).

GS may include the use of familial DNA samples (e.g., the biological parents) for genetic comparison. The use of one or two comparator samples is known as duo or trio testing, respectively. The familial DNA for comparison helps in the interpretation of variants identified by GS by allowing the laboratory scientists to better contextualize identified variants. With this added context, this comprehensive analysis enables prioritization of disease-causing variants leading to a higher diagnostic yield and decreased chance of finding a variant of uncertain significance (VUS) compared to proband-only analysis. An additional yield of about 10% to 15% may be achieved for trio analysis compared to proband-only (Schon et al., 2021; Sullivan et al., 2023) as well as a reduction in VUSs of ˜9% (Rehm et al., 2023). Guidelines prefer the use of trio analysis (Manickam et al., 2021; PLUGS, 2023a). For example, ACMG states “best practice includes familial comparators (“trio”) if available to help contextualize rare variants” (Manickam et al., 2021).

### Example 3: Exome Sequencing in an Outpatient Setting

The majority of rare diseases are serious genetic conditions associated with substantial morbidity and mortality that collectively impact 25 to 30 million people in the United States. These conditions can be challenging to diagnose, often with years-long invasive and costly diagnostic odysseys including the involvement of numerous specialists ordering serial genetic testing and costly medical interventions. Exome sequencing (ES) may be used as a single genetic test providing a timely diagnosis to inform appropriate care.

ES is the analysis of the protein-coding regions of the human genome (called exons), which comprises less than 2% of the entire genome. Prominent professional societies including the American College of Genetics and Genomics (ACMG) have published evidence-based guidelines strongly recommending ES as a first-tier test. Published health economic studies suggest that ES can be cost saving with ACMG guidelines stating using ES as a first or second-tier test “yielded more diagnoses at a lower cost . . . than using standard testing alone.” ES has a diagnostic rate two to three times higher than traditional genetic testing with a diagnostic yield of 36%, reported by a systematic review and meta-analysis. Almost 50% of patients with a positive ES result may have a change in medical management. These modifications included change in medication (new treatment or halting an existing one), alteration to diet, change in planned procedures or surveillance (surgery, imaging, and/or diagnostic studies), referral to specialist, testing of family members, and/or impact on future reproductive planning. Generally, ample evidence supports ES for patients with a suspected rare genetic disease in the outpatient setting.

There are more than 7,000 different diseases that are considered rare. Each condition affects a few hundred to a few thousand people. Collectively, however, they impact about 25 to 30 million people in the United States. About 85% of rare diseases are estimated to be genetic with the majority being serious conditions associated with substantial morbidity and mortality with a considerable medical and financial burden to patients and their families (Tisdale et al., 2021).

These rare diseases can be challenging to diagnose with patients often experiencing years-long diagnostic odysseys, typically involving numerous clinical assessments and investigations that can be invasive and costly (Tan et al., 2017). It has been reported the average time for an accurate diagnosis is approximately 4 to 5 years, but may take over 10 years (Marwaha et al., 2022). The lack of diagnosis or misdiagnoses may result in inappropriate care, lack of targeted treatment, and missed opportunities for interventions that may improve or prevent disease progression (Tisdale et al., 2021).

Establishing an accurate underlying diagnosis based on clinical signs and symptoms is often challenging due to variable presentation and disease course as well as numerous possible genetic causes (Clark et al., 2018; Malinowski et al., 2020). Traditionally, genetic testing was performed serially, using chromosome microarray, single gene analysis, and more recently multi-gene panels, contributing to diagnostic delays or no diagnosis. This approach relies on the subjective assessment of clinicians who may have never encountered a patient with the same constellation of findings, or the findings may be non-specific making a differential diagnosis difficult. However, a different approach is increasingly being utilized in patients with a suspected genetic disease: a broad analysis by a single genetic test, exome sequencing (ES) (Clark et al., 2018).

ES, also known as whole exome sequencing (WES), is the analysis of the protein-coding regions of the human genome (called exons), which comprises less than 2% of the entire genome (Bamshad et al., 2011). The exome contains the genetic sequences currently understood to be most likely to cause clinical phenotypes and disease (Savatt & Myers, 2021). ES allows for detection of sequence level changes (variants) across thousands of genes simultaneously. Additionally, laboratories can report copy number variants (CNVs), providing the ability to also detect multi-exon deletions (loss of regions of genetic material) and duplications (addition of regions of genetic material) as part of the standard ES analysis (Clark et al., 2018; Savatt & Myers, 2021).

ES may be intended for individuals with a suspected rare genetic disorder in which the clinical findings may include congenital anomalies, neurodevelopmental disorders (e.g., epilepsy, developmental delay, intellectual disability, autism spectrum disorders, developmental regression), or dysmorphic features. However, there are additional clinical findings in which ES is indicated as the symptoms of rare disorders are varied and can impact many body systems which may include skeletal, connective tissue, or cardiovascular findings, as well as hearing loss or vision abnormalities (Clark et al., 2018; Retterer et al., 2016).

The standard diagnostic work-up for patients with suspected rare genetic disorders may include radiographs, biopsies, biochemical testing, and serial genetic testing (e.g. chromosomal microarray, single gene testing, multi-gene panels) (Clark et al., 2018; Lavelle et al., 2022). This diagnostic approach is typically a time-consuming and expensive process (Tan et al., 2017). Studies have demonstrated that ES shortens the diagnostic odyssey and can save money. A review of published peer-reviewed studies “suggest that ES can be cost-saving when performed as a first-tier diagnostic test and thus replace serial performance of single-gene, gene panel, and other tests (Smith et al., 2019). Additionally, an evidence-based guideline by American College of Medical Genetics and Genomics (ACMG) stated using ES as a first or second-tier test “yielded more diagnoses at a lower cost . . . than using standard testing alone” (Manickam et al., 2021).

The analytic validation of ES has been published demonstrating a >99% concordance with reference samples and >97% sensitivity of variant calls compared with a commonly available reference data set. (Linderman et al., 2014). In our validation studies, the GeneDx ES test demonstrated a >99.5% concordance with previous identified variants in internal samples and >97% sensitivity of variant calls compared with a commonly available reference data set (Genome in a Bottle) (Krusche et al., 2019).

Classes of clinically relevant genetic variation detectable by ES include single nucleotide variations (SNVs) as well as small deletions, duplications, and insertions. Coverage may vary by laboratory. ES coverage may be 100-120×, which allows detection of additional clinically-significant findings such as copy number variants.

ES has a diagnostic yield two to three times higher than traditional genetic testing (e.g. chromosomal microarray, single gene or targeted panel testing) (Clark et al., 2018; Savatt & Myers, 2021). A systematic review and meta-analysis of 37 studies, which included >20,000 patients with suspected rare disease, reported a diagnostic yield of 36% for ES (Clark et al., 2018). A technology assessment that included 34 studies and >9000 patients with unexplained development disability and/or multiple congenital anomalies reported a 37% (95% confidence interval, 34% to 40%) diagnostic yield for ES (Health Quality Ontario, 2020).

Clinical utility studies based on a meta-analysis have reported a 48% change in medical management for individuals with a positive ES result (Chung et al., 2023). In a systematic evidence review of ES for patients with congenital anomalies (Cas), developmental delay (DD), and/or intellectual disability (ID) initiated by ACMG, 95% of the 167 included studies reported a change to clinical management. These modifications included change in medication (new treatment or halting an existing one), alteration to diet, change in planned procedures or surveillance (surgery, imaging, and/or diagnostic studies), referral to specialist, testing of family members, and/or impact on future reproductive planning (Malinowski et al., 2020). Some of these changes included discontinuation of unnecessary procedures (e.g. diagnostic tissue biopsy), avoidance of certain drugs, or withdrawal of care/start of palliative care (Malinowski et al., 2020). In 2 additional systematic reviews, similar changes in management were reported following ES for patients in select populations. These systematic reviews reported 38% of patients with epilepsy and positive ES having a change in medical management (Sheidley et al., 2022) as well as neurodevelopmental disorders including intellectual disability, developmental delay, and autism in which 30% of with a diagnosis by ES had a change in clinical management and 80% in reproductive planning (Srivastava et al., 2019).

ES may include the use of familial DNA samples (e.g., the biological parents) for genetic comparison. The use of one or two comparator samples is known as duo or trio testing, respectively. The familial DNA for comparison helps in the interpretation of variants identified by ES by allowing the laboratory scientists to better contextualize identified variants. With this added context, this comprehensive analysis enables prioritization of disease-causing variants leading to a higher diagnostic yield and decreased chance of finding a variant of uncertain significance (VUS) compared to proband-only analysis. Publications have reported an additional yield of about 7% to 15% for trio analysis compared to proband-only (Farwell et al., 2015; Lee et al., 2014; Retterer et al., 2016; Sawyer et al., 2016) and a reduction in VUSs of ˜9% (Rehm et al., 2023). Guidelines prefer the use of trio analysis (Manickam et al., 2021; PLUGS, 2023a). For example, ACMG states “best practice includes familial comparators (“trio”) if available to help contextualize rare variants” (Manickam et al., 2021).

### Example 4: Newborn Sequencing

As genomic newborn sequencing (gNBSeq) takes root as a research movement across multiple countries, we report findings from a high-volume commercial laboratory performing sequencing and data analysis for the GUARDIAN (n=12,000) and Early Check (n=2,000) studies. One potential impact of gNBSeq is decreasing time-to-diagnosis. We utilized our large database of over 700,000 exomes and genomes to identify positive findings that would have been reported had gNBSeq been available to patients as newborns. Greater than 21% of patients would have received a diagnosis earlier, on average by more than 8 years. gNBSeq has the potential to significantly decrease time-to-diagnosis, which is particularly critical for diagnoses with associated treatments and/or interventions.

Various takeaways were identified via this study.

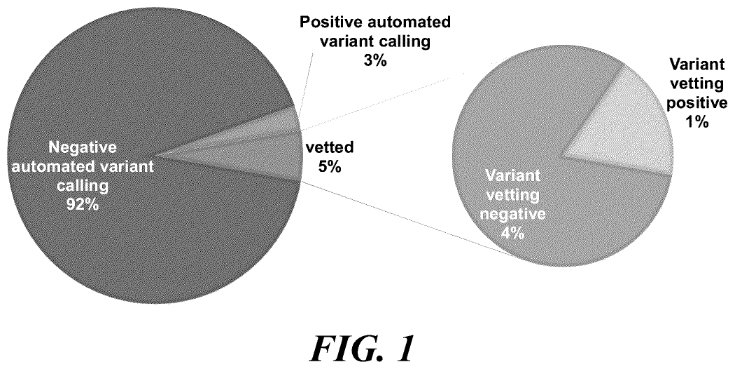

Interpretation of gNBSeq is heavily dependent on having a robust database of clinically significant variants to reduce the burden of analysis. This can facilitate scalability via automation of common reportable variants. Our internal database enabled us to limit the average gNBSeq review to ˜10 variants, with 95% of cases automatically called (automated variant calling) (see FIG. 1).

Phasing common clinically significant alleles is necessary due to short read sequencing limitations. Expertise in technical evaluation and understanding sequencing data outputs are necessary to inform variant interpretation and deliver a more definitive diagnosis. CFTR variants that are almost exclusively inherited together (in cis, same haplotype) lead to frequent false positives. We modified our reporting strategy after the first 1000 cases by integrating guidance from the CFTR2 database, eliminating VUS reporting, and adjusting our reporting guidelines to only report common haplotype alleles when seen with a second pathogenic allele. G6PD haplotypes have functional consequences requiring specific interpretation and reporting language. Establishing haplotype classification was able to reduce burden on analysis, while simultaneously expediting reporting. Leveraging internal and external resources (e.g., gnomAD) we were able to establish a workflow for designating complex alleles to reduce false positives (e.g., SLC12A3).

Genes with variable expressivity and/or reduced penetrance present a challenge requiring expert gene knowledge. GJB2 has pathogenic alleles with a wide range of disease onset, therefore traditional newborn screening approaches have a lower false positive rate for early-onset sensorineural hearing loss than gNBSeq. This gene was removed in V2 of GUARDIAN. SCN1A has variable expressivity and no alternative screening methodology, leading to a need for high-throughput functional studies to complement traditional criteria applied with the ACMG classification system. Our extensive gene expertise informs variant interpretation for all ion channel genes, reducing the false positive rate

Genes with dual disease/inheritance require nuanced interpretation and may hamper scalability. A highly-curated gene-disease knowledgebase is essential. Without expert gene list curation prior to testing, AD/AR genes with unknown mechanisms lead to overreporting of carrier status and therefore false positives. Not all diseases associated with a gene may be appropriate for gNBSeq. For example, MITF-related Waardenburg syndrome has a common pathogenic variant associated with MITF-related cutaneous malignant melanoma.

### Summary and Conclusions

Our experience with the largest gNBSeq cohort, with diverse multi-site implementation strategies, has enabled us to share specific lessons learned and positions GeneDx as a laboratory leader for future gNBS progress and initiatives. By innovating and improving genomic results for automated variant calling, phasing, variable expressivity, reduced penetrance, and gene variants associated with more than one disease, we are moving the needle forward for understanding the scalability and clinical utility of gNBSeq.

### Example 5: Genome and Exome Sequencing for Health Systems

In some examples, electronic health databases such as electronic health record (EHR) databases interface with genomic screening platforms and systems disclosed herein. Information such as test results and testing orders contained in EHRs are retrieved via an integration platform. The retrieved test results or test orders, or both, are utilized to determine an applicable genetic test for a subject based on the subject's EHRs. In some examples, an order is placed for the applicable genetic test by an integration platform based on the retrieved or received health information from the EHRs. The genetic tests include, for example, exome sequencing (ES), whole genome sequencing (WGS), rapid whole genome sequencing (rWGS), or any combination thereof.

The integration of genetic testing with EHRs, in some examples, includes health systems and healthcare network softwares accessing genetic testing within a native EHR system. For example, health system infrastructure can access genomic testing options within a native EHR system using an integration platform or plugin. For example, healthcare providers and other users of health systems receive an indication of appropriate or recommended genomic tests or genomic testing kits for a patient based on information in the patient's EHR. The information in the patient's EHR can be diagnostic information, laboratory test result information, demographic information, identification information, notes from healthcare providers, or any other type of health information.

A health system infrastructure also receives results of the genomic testing. The genome testing options, such as ES, WGS, and rWGS, are integrated with health systems such as EHR databases so that results of the genomic testing performed are integrated into the EHR. The integration of genomic testing results into the native EHR system of a healthcare provider, such as a health system, can be performed via an integration platform or an integration plugin. The genetic testing results for a patient are integrated with other laboratory results in the EHR, and are not sequestered in a separate location in the EHR of the patient. The integration of genomic test results with other laboratory results can, for example, make the genomic test results easier to visualize and compare with other testing results.

Integration of genomic testing and native EHR systems of health systems include integration of genomic testing recommendations and results into existing workflows of the EHR systems and health systems. The genomic testing recommendations or genomic testing results, or both, can be integrated across multiple platforms, for example. Mechanisms or elements of graphic user interfaces (GUIs), or both, for ordering genomic testing and viewing results of genomic testing can be integrated into existing workflows and existing GUIs of health systems such as EHR systems.

For example, tools, interfaces, and platforms can be used to initiate a communication between one or more healthcare providers, or other members of a health system, with a person related to the genetic testing. For example, healthcare providers can connect with developers and technical experts of the genomic testing kits to discuss results or recommended options of various genomic tests.

The data relating to the genomic tests, such as genetic testing result data or data concerning an appropriate genetic test type for a patient, is structured. A format of the data can be modified from the genetic testing output data to the structured data format. The structured data format is configured to be compatible with the EHR systems or health systems data types. The structured data format is configured to, for example, integrate with the EHR system data types or health systems data types in a single section. The structured data format of the genomic data can be retrieved and modified to facilitate research and quality improvements. This can include, for example, modifying data results of the structured data type to integrate additional data or variables.

Reports originating from or generated by the genomic tests or genomic testing platforms can be integrated into the EHR systems, for example integrated within EHR data. Data of the genomic testing reports can be integrated in medical records of a patient.

Integration of genomic testing systems, platforms, and kits can include communication between a healthcare provider or a user of the healthcare system to request reanalysis of a sample of a patient from an entity relating to the genomic testing systems, kits, platforms, or protocols. The reanalysis can include reanalysis of one or more specific genes, reanalysis of one or more specific variants, or both. Reanalysis can include reanalysis of an entire genome of an individual, or targets of the genomic test. A healthcare provider or user of a healthcare system can submit additional or new information about a health condition, diagnosis, or symptom of the patient to a health system or EHR system, which can result in a modification of a recommended genetic test, a genetic testing result, or a genetic testing report, an initiation of a reanalysis, or any combination thereof.

### Example 6: Cerebral Palsy (CP) Diagnostics

Cerebral palsy (CP) is the most prevalent childhood motor disability, is nonprogressive, and often associated with other neurodevelopmental disorders. Birth trauma was historically felt to be a major cause of CP. However, recent findings suggest that diagnostic genomic variants are identified in up to 1 in 3 individuals with CP, supporting the use of exome and genome sequencing (ES/GS) as a first-tier test.

Large, deidentified genetic testing datasets were utilized to determine the diagnostic rate of exome sequencing (ES) and genome sequencing (GS) for individuals with CP. Additionally, genetic heterogeneity was explored for individuals with CP who were found to have diagnostic genetic findings.

For example, the molecular diagnostic yield of ES/GS-based testing was reviewed for 9,141 individuals with a clinical diagnosis of CP who did not opt out of research and had genetic testing at a commercial laboratory between 2015-2025. Individuals were identified as having Likely Pathogenic or Pathogenic (L/PATH) variants in known disease genes associated with the reported phenotype provided for the proband, as categorized using American College of Medical Genetics and Genomics (ACMG) classification criteria. Evaluation was performed of genetic heterogeneity and detection rates based on neurodevelopmental and congenital comorbidities reported at the time of testing. Analyses excluded individuals with only multi-gene pathogenic copy number variants reported.

The ES/GS identified diagnostic variants in 2,566 individuals (28.1%). Diagnostic L/PATH variants in 794 genes were reported. There was genetic heterogeneity, with 48.7% of the reported genes having a finding in a single individual. A small percentage of individuals (3.4%) had a dual diagnosis with findings in either two genes (n=52) or a single gene and multi-gene copy number variant (n=34). On average, individuals with a diagnostic finding had 2.09 comorbidities including neurodevelopmental delay, seizure, intellectual disability (ID), autism and hearing impairment, in order of prevalence. The diagnostic rate was highest for ID as a single comorbidity (37.5%), increased to 39.2% for individuals with both ID and autism and was 14.3% when none of the five comorbidities were reported. The six tier I genes identified as highly likely to be associated with CP using a gold standard definition were among the most common genes in the cohort: COL4A1 (n=49), SPAST (n=29), TUBA1A (n=26), GNAO1 (n=24), KIF1A (n=19), and ATL1 (n=18). In addition to COL4A1, variants in COL4A2 were also reported. Both genes are associated with brain small vessel disease and risk of intracerebral hemorrhage [OMIM: 120130]. While some individuals had a history of brain hemorrhage that could have contributed to their CP, the majority had CP without a known event. Other common findings included individuals with variants in CTNNB1 (n=45) and DDX3X (n=40), which have been reported in multiple individuals with CP in prior studies. Some diagnostic findings were for conditions not associated with CP but with progressive, degenerative, or biochemical phenotypes such as spinal muscular atrophy, ataxia-telangiectasia, or dopa-responsive dystonia.

A diagnosis of CP can evolve with other emerging clinical features, making genetic testing a tool with potential to elucidate the molecular diagnosis and clarify the diagnosis. Early ES/GS can contribute to a more precise diagnosis and can inform recurrence risk, addressing causes and comorbidities of CP.

### Example 8: Exome Testing Reanalysis

Genetic testing can sometimes result in unclear, uncertain, or negative results. These results can be addressed with, for example, broad testing and reanalysis options. Multi-step methods can determine when reanalysis and broad testing should be performed, and what types of these should be performed, upon receiving an unclear or negative result. Negative result can include no genetic variants being identified in a sample of a subject.

Upon receiving a negative or unclear result, systems and methods can address several steps of questions and procedures. For example, information about the unclear or negative result can be determined. The gene subject to the genetic testing or estimated to have a variance determined by the genetic testing is identified. The variant is then identified. The laboratory that had performed the test with the negative or unclear result was then identified. The type of test performed was then identified. The date the test was performed, the time the test was performed, the season the test was performed, the month the test was performed, the year the test was performed, or the day of the week the test was performed, or any combination thereof, was determined. Next, reanalysis and broad testing, such as exome sequencing, was pursued. This reanalysis and broad exome sequencing was pursued based on the information determined concerning the gene, variant, testing laboratory, test type, date of testing, or any combination thereof.

Additionally, communication can be initiated with gene-specific groups, disease-specific groups, genetic testing groups, or a wider community, or any combination thereof. This communication can be initiated based at least in part on the information determined in the reanalysis and broad exome sequencing pursuit determination.

Next, reanalysis steps can be performed depending on result type. Reanalysis can be performed for uncertain results. Reanalysis can be performed for generally uncertain test results, results uncertain as to the gene, or results uncertain as to the variant. Reanalysis can be performed for the entire test or for one or more genes that were subject to uncertainty, or for one or more variants that were subject to uncertainty in the result, or any combination thereof. Reanalysis for negative results can be performed for generally uncertain test results. Reanalysis for negative results can be performed on the test at large. Broad testing can also be pursued.

Reanalysis is pursued or recommended for exome sequencing. Reanalysis is performed after two to three years, for example. Reanalysis can be performed after one month, two months, three months, four months, five months, six months, seven months, eight months, nine months, ten months, eleven months, twelve months, about one year, about two years, about three years, about four years, about five years, about six years, about seven years, about eight years, about nine years, about ten years, or more than about ten years after the initial testing.

Healthcare providers can request reanalysis for a patient. Healthcare providers can request an exome reanalysis, for example. Healthcare providers can inform a testing laboratory of any new symptoms or features of the patient. Information about new symptoms or features of the patient can be integrated into genomic testing parameters in genomic testing systems, platforms, or kits, or any combination thereof. Healthcare providers can also communicate newly identified family members of the patient with the same features. Information about family members of the patient with similar or the same features as the patient can be integrated into genomic testing parameters in genomic testing systems, platforms, or kits, or any combination thereof. A new candidate gene finding can be determined for the patient upon reanalysis. A new positive result can be generated by the reanalysis. New insights can be generated by the reanalysis. New insights can be generated with or without a new positive result.

Gene reclassification can be pursued through genomic reanalysis. For candidate genes or genes of uncertain significance, genomic reanalysis testing can be performed to reclassify the genes. A healthcare provider can communicate with a laboratory to determine if a gene is considered a candidate gene or gene of uncertain significance. Healthcare providers can inform a testing laboratory of any new symptoms or features of the patient. Information about new symptoms or features of the patient can be integrated into genomic testing parameters in genomic testing systems, platforms, or kits, or any combination thereof. Healthcare providers can also communicate newly identified family members of the patient with the same features. Information about family members of the patient with similar or the same features as the patient can be integrated into genomic testing parameters in genomic testing systems, platforms, or kits, or any combination thereof. Significance of downstream or epigenetic effects, or characteristics of the patient can be determined and linked to the candidate gene, so that the gene is no longer of uncertain significance.

Additionally, for example, variants can be reclassified. Genomic reanalysis can be performed for variant reclassification. Alternatively or additionally, data of prior genomic analysis can be reanalyzed or reclassified to reflect a reclassification of one or more variants of the genomic testing results. For example, a healthcare provider can communicate a query concerning a change in classification of one or more variants. The change in classification can occur after a previously generated genomic testing results report. Healthcare providers can inform a testing laboratory of any new symptoms or features of the patient. Information about new symptoms or features of the patient can be integrated into genomic testing parameters in genomic testing systems, platforms, or kits, or any combination thereof. Healthcare providers can also communicate newly identified family members of the patient with the same features. Information about family members of the patient with similar or the same features as the patient can be integrated into genomic testing parameters in genomic testing systems, platforms, or kits, or any combination thereof. Genomic variants can be reclassified based on information concerning new symptoms or features of the patient, or similar or the same symptoms or features of the patient's family members, or genomic reanalysis results, or any combination thereof.

Variant reclassification can include a determination of whether the reclassification of the variable is likely not clinically actionable, or if the reclassification of the variant is likely clinically actionable. The variant reclassification can be determined to be one of several groups involving a judgment of the benign or pathogentic nature of the reclassification of the variant. The groups can comprise a spectrum between benign and pathogenic. These groups can comprise benign, likely benign, variant of uncertain significance, likely pathogenic, or pathogenic.

The reclassification of the variant can be evaluated to be not clinically actionable if it is benign, likely benign, a variant of uncertain significance, or even possibly pathogenic. The reclassification of the variant can be evaluated to be clinically actionable if it is likely pathogenic or pathogenic.

The reclassification of the variant can be evaluated and determined to be not clinically actionable depending on a series of factors and characteristics. The series of factors and characteristics can include that the reclassified variant has an unusually high frequency in the population, the reclassified variant is homozygous in unaffected individuals, strong functional evidence of normal protein function with the reclassified variant, silent or intronic variant(s) outside the consensus splice site of the reclassified variant, resulting in no impact on RNA splicing, or any combination thereof, or example. The series of factors and characteristics can also include the reclassified variant having a usually absent or low frequency in the population, weak functional evidence such as normal protein function with the reclassified variant, weak functional evidence such as minimal disruption of protein function with the reclassified variant, or any combination thereof, for example. The series of factors and characteristics can also include a silent or intronic variant in the consensus splice site of the reclassified variant resulting in little potential impact on RNA splicing, or no impact on RNA splicing, a missense variant with no clinical or functional data, weak segregation with disease, or some case reports in unrelated affected individuals.

The reclassification of the variant can be evaluated and determined to be clinically actionable depending on a series of factors and characteristics. The series of factors or characteristics can include if the reclassified variant is usually absent or low frequency in the population, strong functional evidence that protein function is disrupted by the reclassified variant, indication that the reclassified variant is in the AG/GT dinucleotide and impacts RNA splicing, or any combination thereof, for example. The series of factors or characteristics can also include if the reclassified variant is a nonsense or frameshift variant, resulting in suspected loss of protein, if the reclassified variant has a strong segregation with disease, or multiple case reports of the reclassified variant in unrelated affected individuals, or any combination thereof, for example.

A report on the impact of variant reclassification and reanalysis factors can be generated by systems or platforms associated with the genomic tests. The report can be a population-level aggregate report or a report of an individual patient or group of patients. The report can be integrated with the EHR of the patient or multiple EHRs of a group or population of patients. The report can be integrated using a medical system integration platform.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-14. (canceled)

15. A method comprising:
(a) assaying a bodily sample from a subject to generate sequencing information;
(b) computer processing the sequencing information using a gene-phenotype knowledge base to identify a prospective genetic variation and a phenotype associated with the prospective genetic variation;
(c) determining a pathogenicity of the prospective genetic variation,
wherein determining the pathogenicity comprises computer processing the prospective genetic variation and the phenotype identified in (b); and
(d) generating a human interpretation of a clinical significance of the pathogenicity determined in (c), wherein the human interpretation comprises applying a set of pre-determined criteria to the determining in (c).

16. The method of claim 15, wherein the bodily sample is a buccal sample, a blood sample, a saliva sample, a urine sample, a cell sample, or a tissue sample.

17. The method of claim 15, wherein (a) comprises extracting DNA from the bodily sample.

18. The method of claim 17, further comprising preparing a sequencing library from the DNA.

19. The method of claim 17, wherein (a) comprises subjecting the DNA to a sequencing reaction.

20. The method of claim 19, wherein the sequencing reaction comprises a whole genome sequencing reaction, or an exome sequencing reaction, or both.

21. The method of claim 19, further comprising subjecting the DNA, or derivatives thereof to one or more pull down probes.

22. The method of claim 15, wherein the subject is less than 18 years old, 17 years old, 16 years old, 15 years old, 14 years old, 13 years old, 12 years old, 11 years old, 10 years old, 9 years old, 8 years old, 7 years old, 6 years old, 5 years old, 4 years old, 3 years old, 2 years old, or 1 year old.

23. The method of claim 15, further comprising obtaining data relating to the phenotype of the subject is based at least in part on medical records or clinical notes.

24. The method of claim 15, wherein the prospective genetic variation comprises one or more of: a substitution, an insertion, a deletion, a single nucleotide variation, a copy number variation, a mobile element insertion (MEI), or a Uniparental disomy (UPD), or any combination thereof.

25. The method of claim 15, further comprising prior to (c), performing an orthogonal assay to confirm a presence of the prospective genetic variation.

26. The method of claim 25, wherein the orthogonal assay comprises a chromosomal microarray analysis (CMA), exon-level ‘exon-array’ microarray, qPCR, multiplex ligation-dependent probe amplification (MLPA), or a Sanger sequencing assay.

27. The method of claim 15, wherein the prospective genetic variation is classified as likely benign, benign, likely pathogenic, pathogenic, or uncertain significance.

28. The method of claim 27, wherein a genetic variation classified as likely benign indicates at least about a 90% certainty of benignity.

29. The method of claim 27, wherein a variant classified as benign indicates at least about a 99% certainty of benignity.

30. The method of claim 27, wherein a variant classified as likely pathogenic indicates at least about a 90% certainty of pathogenicity.

31. The method of claim 27, wherein a variant classified as pathogenic indicates at least about a 99% certainty of pathogenicity.

32. The method of claim 15, wherein the processing comprises using a trained machine learning algorithm.

33. The method of claim 15, wherein the processing outputs one or more scores indicative of strength of association of the prospective genetic variation with the phenotype associated with the prospective genetic variation.

34. The method of claim 15, wherein the gene-phenotype knowledge base comprises data derived from two or more related individuals, or published literature, or both.

35. The method of claim 15, wherein the set of pre-determined criteria is based at least at least in part on one or more of: a frequency of the prospective genetic variation in affected and unaffected populations of a disease or disorder, a variant type, a disease mechanism, segregation patterns of one or more loci, analysis of functional studies, analysis of case studies, or analysis of cohort studies, or any combination thereof.

36. The method of claim 15, wherein the set of pre-determined criteria is based at least upon data from the subject and a data from a mother or father of the subject.

37. The method of claim 15, wherein the method is performed in less than 7 days.

38. The method of claim 15, wherein the method is performed in less than 10 days.

39. The method of claim 15, wherein the method is performed with an accuracy of at least about 90%.

40. The method of claim 15, wherein the human interpretation further confirms the phenotype associated with the prospective genetic variation.

41. The method of claim 40, further comprising, based at least on the presence of the prospective genetic variation and the phenotype associated with the prospective genetic variation, determining that the subject has an elevated risk of having a disease, disorder, or condition.

42. The method of claim 41, wherein determining that the subject has the elevated risk of having the disease, disorder, or condition comprises a sensitivity of at least about 90%.

43. The method of claim 41, wherein determining that the subject has the elevated risk of having the disease, disorder, or condition comprises a specificity of at least about 90%.

44. A method comprising:
(a) obtaining training information comprising a set of genes and a set of phenotypes;
(b) computer processing the training information to identify a prospective genetic variation and a phenotype associated with the prospective genetic variation;
(c) generating a human interpretation of a clinical significance of an association between the prospective genetic variation and the phenotype,
wherein the human interpretation comprises applying a set of pre-determined criteria to the identifying in (b); and
(d) incorporating the association into a gene-phenotype knowledge base, based at least in part on the human interpretation in (c).
